(19) United States
(12) Patent Application Publication
(10) Pub. No.: US 2021/0035672 A1
(43) Pub. Date: Feb. 4, 2021

(54) METHOD AND APPARATUS FOR INDIVIDUALIZED ADMINISTRATION OF MEDICAMENTS FOR DELIVERY WITHIN A THERAPEUTIC RANGE

(71) Applicant: University Of Maryland, Baltimore, Baltimore, MD (US)

(72) Inventors: Jogarao Gobburu, Herndon, VA (US); Vijay Ivaturi, Baltimore, MD (US)

(21) Appl. No.: 17/044,097

(22) PCT Filed: Apr. 5, 2019
(86) PCT No.: PCT/US19/26069

§ 371 Date: Sep. 30, 2020

Related U.S. Application Data
(60) Provisional application No. 62/653,039, filed on Apr. 5, 2018.
(60) Provisional application No. 62/652,990, filed on Apr. 5, 2018.

(51) Int. Cl. G16H 20/10; G16H 50/70; G16H 70/40; G16H 10/60; G06N 20/00

(52) U.S. Cl. CPC G16H 20/10; G16H 50/70; G06N 20/00; G16H 10/60; G16H 70/40

(57) ABSTRACT

Techniques for generating a dosing protocol for an individual include a continuous multivariate model for dose response to a medicament, the model having a population parameter that characterizes a population of individuals and a random parameter based on a distribution of observed values in the population. A first dose based on a naive most probable value for the random parameter; a probability that the first dose is therapeutic; and, a set of one or more times to sample the subject for the therapeutic effect based on the probability, are all determined using the model. The subject is sampled to obtain measured values of the therapeutic effect. A subject-specific most probable value for the at least one random parameter is determined based on the model and measured values. A second dose and timing therefor are determined based on the subject-specific most probable value.

### CROSS-REFERENCE TO RELATED APPLICATIONS

This application claims benefit of Provisional Appln. 62/652,990, filed Apr. 5, 2018, and Provisional Appln. 62/653,039, filed Apr. 5, 2018, the entire contents of each of which are hereby incorporated by reference as if fully set forth herein, under 35 U.S.C. § 119(e).

### BACKGROUND

Different patients process administered drugs differently; and for some drugs or for some neonates, or some combination, the variability in delivery of drug concentration for the same amount administered can have a strong bearing on the effectiveness of the drug. As used herein, the term medicament means any material administered to a subject for therapeutic effect, including drugs and other biological agents, such as large molecules, nucleic acids, viruses and bacteria. In general, one can say that, for some medicaments, careful and precise management of administration and delivery is of critical importance. The process of managing administration and delivery of drugs has been called therapeutic drug management (TDM) in the health care industry. Here we use the term TDM to refer to the management of both drugs and other medicaments.

Vancomycin is one of the more frequently used antibiotics in neonatal intensive care units (NICU). Underexposure (defined as minimum concentration {measured or inferred by rate of elimination} before the next dose is administered, and abbreviated Ctrough, <10 mg/L) can lead to treatment failure and/or resistance development, and overexposure (Ctrough>20 mg/L) to acute nephrotoxicity. To balance these risks, Vancomycin is routinely subjected to TDM. Unlike Vancomycin treatment in adults, which is reasonably standardized, numerous dosing nomograms have been proposed for neonates (24), in which dose varies with weight or other covariate. However, in fact, even with such nomograms, prospective and retrospective studies across the US and Europe have shown as few as 37% of neonates initially achieve recommended target concentrations. Even more concerning, though TDM is frequently used, analyses still report final target attainment statistics around only 45%. Comparison of nomogram performance between hospitals is difficult. Adoption of nomograms across hospital systems is not well-publicized, and details about clinician therapeutic drug management (TDM) interventions are non-existent in publications. Furthermore, comparison of nomogram performance in isolation is tricky, as nomogram development is also guided by hospital protocols, historical precedent, and physician preferences. For example, a nomogram developed for a hospital that has established a therapeutic target range of 5-25 mg/L will understandably provide lower dose recommendations than one targeting 15-25 mg/L.

### SUMMARY

Techniques are provided for determining, during treatment of a subject, individualized protocols for administering medicaments, which have increased chance to be maintained in the subject within a therapeutic range.

In a first set of embodiments, a method for generating a dosing protocol for an individual, includes receiving on a processor first data that indicates, for dose response to a medicament, a continuous multivariate model with at least one population parameter based on a single value that characterizes a population of individuals and at least one random parameter based on a distribution of values for individuals in the population. The method also includes administering to a subject a first dose of the medicament based on the model and a naive most probable value for the at least one random parameter. Furthermore, the method includes determining, automatically on the processor based on the model, a probability that the first dose will produce a therapeutic result and a set of one or more times to sample the subject for corresponding measures of the therapeutic effect based on the probability. Still further the method includes sampling the subject at the set of one or more times to obtain corresponding values of the corresponding measures of the therapeutic effect. Even further, the method includes determining, automatically on the processor based on the model, a subject-specific most probable value for the at least one random parameter based on the corresponding values. Yet further still, the method includes determining, automatically on the processor based on the model, a second dose and timing therefor based on the subject-specific most probable value.

In some embodiments of the first set, the method further includes administering to the subject the second dose of the medicament.

In various embodiments of the first set, at least one random parameter is based on a distribution of values in electronic health records (EHR); or, the at least one random parameter is at least one of clearance or volume; or, the medicament is vancomycin.

In some embodiments of the first set, the at least one population parameter is learned based on machine learning and a training set based on electronic health records (EHR). In some of these embodiments, the machine learning is based on a neural network.

In some embodiments of the first set, the continuous multivariate model includes at least one covariate variable and the method further comprises receiving on a processor second data that indicates a corresponding value for the at least one covariate variable. In some of these embodiments, the at least one covariate variable is at least one of weight or age or gender. In some of these embodiments, the medicament is vancomycin and the at least one covariate variable includes estimated glomerular filtration rate.

In some embodiments of the first set, a probability of the subject-specific most probable value is greater than a probability of the naive most probable value.

In other sets of embodiments, a computer-readable medium or a system is configured to perform one or more steps of one or more of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example pharmacokinetic and pharmacodynamics system model, used according to an embodiment;

FIG. 2A is a block diagram that illustrates example flow structure of a generic neural network computation;

FIG. 2B is a plot that illustrates example activation functions used to combine inputs at any node of a neural network;

FIG. 3 is flow diagram that illustrates an example method for objectively and automatically determining model parameters and updating a model using medicament response observations for an individual during treatment, according to an embodiment.

FIG. 4A through FIG. 4F are plots that show dose administration times, model results and measurements starting at time of first dose for six representative patients, according to an embodiment;

FIG. 5 is a block diagram that illustrates a computer system upon which an embodiment of the invention may be implemented; and,

FIG. 6 illustrates a chip set upon which an embodiment of the invention may be implemented.

### DETAILED DESCRIPTION

A method and apparatus are described for determining, during treatment of a subject, individualized protocols for administering medicaments, which have increased chance to be maintained in the subject within a therapeutic range. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of precision dosing of vancomycin in neonates. However, the invention is not limited to this context. In other embodiments, a corresponding precision therapeutics toolset would be directly applicable to all medicaments that benefit from TDM. Prediction models other than those for precision dosing can be incorporated, e.g., to assess appropriateness of palliative chemotherapy in patients with metastatic cancer with dismal prognosis by predicting survival; to use appropriate anti-diabetic therapy in patients with type 2 diabetes of varied pathophysiology; or to predict cardiovascular event risk in a given time period.

### 1. OVERVIEW

Biologically and pharmacologically plausible systems models such as Pharmacokinetic (PK) and Pharmacodynamic (PD) (PK/PD) models are tailored for concentration levels of a dose in individual subjects. The systems models for precision therapeutics are highly non-linear and require multiple levels of uncertainty and variation, leading to the use of non-linear mixed effects (NLME) models. NLME for statistical inference is demonstrated in a large literature of applied statistics. Moreover, the form of NLME models limits the types of usable patient data to easily provided quantities such as age, sex, and weight. The systems models proposed here with NLME are used in conjunction with modern machine learning algorithms such as deep (convolutional) neural networks, density-based clustering on diffusion mapped data, or gradient-boosted regression trees. This pushes well beyond modeling for the averages to a space filled with complex multi-dimensional healthcare information with both fixed and random effects models. Such modeling attempts to capture a therapeutically significant subset of the underlying complex pharmacological mechanisms that can be used to individualize therapeutics one patient at a time.

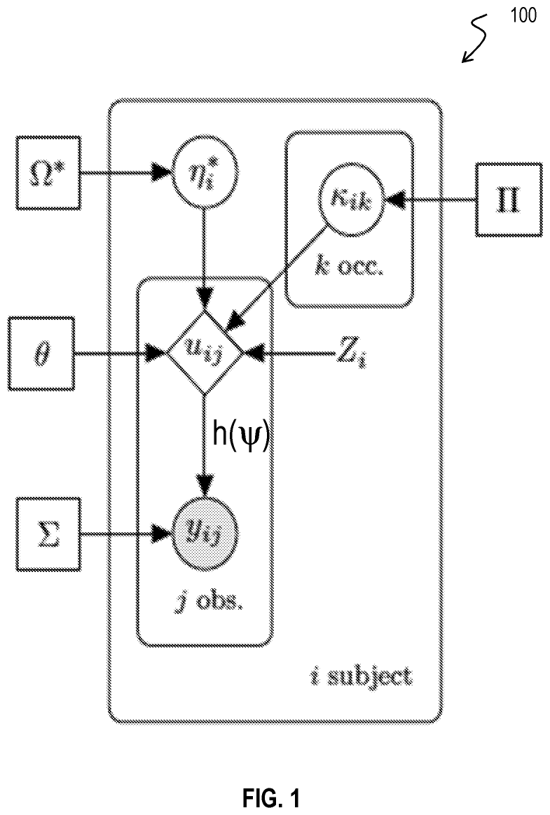

As used herein, a subject is a human or animal patient or healthy recruit or surrogate or a simulation of same. FIG. 1 is a block diagram that illustrates an example pharmacokinetic and pharmacodynamics system model 100, used according to an embodiment. In FIG. 1, the rectangle nodes denote parameters, circles denote random quantities which are either latent (unfilled) or observed (filled), diamonds are deterministic given the inputs, and nodes without a border are constant. The system model 100 describes the evolution of one or more observable medicament levels yᵢⱼ for individual i at observation time elements j. Such a system is defined by three components: random effects; dynamics; and observations.

A random effect, represented by the symbol ηᵢ, captures the inter-individual variability (IIV) for individual i within a population of subjects. The variable ηᵢ, represents one or more random effects for individual i and thus, in general, is a vector. The distribution of this effect in the whole population is parametrized by Ω. Sometimes, one captures the inter-occasion variability (IOV) effect denoted by κᵢₖ at each occasion k for individual i, e.g., when an individual patient visits a hospital on multiple occasions for a tissue sample to determine medicament levels (such as a blood draw to determine drug concentration value in the blood at those times). κᵢₖ represents one or more inter-occasion random effects for individual i and thus, in general, is a vector. The distribution of the inter-occasion effect over the whole population is denoted by Π. The most common choice for random effect components is some sort of multivariate normal distribution N with zero mean of the form ηᵢ=N(0, Ω) and κᵢₖ=N(0, Π).

Dynamics control the time evolution of the system. A dynamic variable uᵢⱼ represents the unobserved (latent) dynamics of the model that unfold at a sequence of time points tᵢⱼ for individual i and time element j. uᵢⱼ represents one or more latent dynamic variables for individual i and thus, in general, is a vector These dynamics are provided as the solution to a system of differential equations including as parameters one or more nonrandom parameters in a vector θ, the one or more random effects ηᵢ, and zero or more subject-specific values for covariates variables in vector Zᵢ. Co-variates Zᵢ is a vector of zero or more observable characteristics of individual i, which are known to vary with yᵢⱼ that is being modeled. The functional relationship among these variables is expressed in Equation 1a.

uᵢⱼ=uᵢ(tᵢⱼ)=u(tᵢⱼ;θ,ηᵢ;Zᵢ) (1a)

The actual differential equations represented by Equation 1 have a form depending on the medicament being modeled.

Observations yᵢⱼ is a vector of one or more actual observed response variables (also called dependent variables). These are related to the dynamic variables uᵢⱼ by an observation function ψ; and, assumed to have some distribution which depends on uᵢⱼ and zero or more additional random parameters, such as random errors, represented by the vector σ whose distribution over the whole population is denoted by Σ, as given by Equation 1b.

yᵢⱼ=h(ψ(uᵢⱼ),σ) (1b)

A nonlinear mixed effect (NLME) model output is determined as follows. From the distributions (Ω, Π, Σ) the random variables (ηᵢ, κᵢₖ, σ) are defined. The covariates Zᵢ for each individual are given, as are the parameter values θ for the population. These pieces then define the parameters for the dynamical model for a particular set of differential equations represented by Equation 1a, which is solved (analytically or numerically). Then the observation function ψ is applied, then the prediction model h with error values ϵ_{L} (having standard deviation σ) is applied as represented in Equation 1b, resulting in modeled observations, e.g., for a simulation. As a result, the modeling process can be thought of as the map from inputs to outputs given by an expression labeled Equation 1c.

(θ,Zᵢ,Ω,Π,Σ)→yᵢⱼ=yᵢ(tᵢⱼ) (1c)

For training and for individualized administration of a medicament, the interest is in the inverse problem of learning more about the model given data of real observations, represented by the symbol dᵢⱼ=dᵢ (tᵢⱼ). The desire is to learn one or more of θ, Zᵢ, and Ω Π, Σ, (thus effectively the amount of randomness for the random effects, random occasions and random measurements) or more explicitly at least random draws from those last three distributions ηᵢ, κᵢₖ, and ϵᵢ. This inverse problem, summarized as finding the parameters such that ∥d−y∥ is minimized, is known as the estimation problem. This minimization is performed by finding the parameters that maximize the population likelihood or via Maximum A Posteriori (MAP) estimation. Common methods which approximate the population likelihood include: 1) First order conditional estimation 2) Laplace approximations 3) Expectation maximization (sampling-based methods). In addition, more accurate posterior simulation algorithms such as Hamiltonian Monte Carlo, Stochastic Approximation Expectation-Maximization (SAEM), and Approximate Bayesian Computation (ABC) are used for parameter estimation in some embodiments.

In PK compartment models, drug concentrations (C) in the compartments are equal to the amounts (A) divided by volumes (V). For two compartments, C1=A1/V1 and C2=A2/V2. Drug concentration in the central compartment (C1) is equal to the concentration in the plasma (CP). Clearance (CL, in units of liters, L, per hour) is often used instead of the fractional rate constants k_{α} (in units of per hour, not to be confused with occurrence k). In pharmacokinetics, the distribution volume is given in volume units (L), and rate constants can be represented as the ratio of clearance and distribution volume, e.g., k_{α}=CL/V. In the case of oral administration of the drug, at time t=0 the amount of drug in the central and peripheral compartments is zero (A1(0)=A2(0)=0), and the initial amount in gastrointestinal tract (effective dose) is: AGI(0)=D×S×F, where D is the administered dose of the drug, S is the salt factor (fraction of administered dose that is made up of pure drug), and F is the bioavailability factor (fraction of dose that reaches the systemic circulation). Parameters often used in PK models include D=dose, τ=dosing interval, CL=clearance, Vd=volume of distribution, kₑ=elimination rate constant, k_{α}=absorption rate constant, F=fraction absorbed (bioavailability), K₀=infusion rate, T=duration of infusion, and C=plasma concentration.

In example embodiments, one or more of these parameters might be described or affected by a distribution about a typical value of random values for different individuals.

An example embodiment illustrates a one-compartment PK model for a drug given via oral administration as multiple doses (AGO, also called herein the “depot” such as the gut) and the resulting concentration in the circulating blood or plasma tissue (central). The ordinary differential equations (ODE) used to determine the form of Equation 1a for this embodiment are given by Equations 2 and 3.

$$\begin{matrix}\frac{d [ Depot ]}{dt} = - {k}_{a} [ Depot ] & ( 2 ) \\ \frac{d [ Central ]}{dt} = {k}_{a} [ Depot ] - \frac{C L}{V} [ Central ] & ( 3 )\end{matrix}$$

where the equation is determined by pharmacokinetic principles based in mass-action laws, and where CL and V are clearance and volume. In this case, uᵢ is the vector of drug concentrations in an individual, expressed by Equation 4.

$${u}_{i} = ( \begin{matrix}Depot \\ Central\end{matrix} ) \qquad ( 4 )$$

In this formulation, ψ is a vector-valued function for the right-hand side of the ODE because the dynamic variable uᵢ is concentration, which is also measured (observed) at the occurrences κᵢₖ. The ODE's parameters (both random and fixed) are collected in vector gᵢ for the individual as given by Equation 5.

$${ℊ}_{i} = [ \begin{matrix}{k}_{a} \\ C L \\ V\end{matrix} ] \qquad ( 5 )$$

In addition to the models, the relation between the ODC coefficients and the model's random and non-random parameters and covariates are also given. For example, in some embodiments in which CL depends on two covariates weight (wt) and gender (sex) of individual i with some random variability denoted by η_{CL}, such a relationship can be given by Equation 6 in terms of four population parameters θ₁ through θ₄.

$${ℊ}_{i} = [ \begin{matrix}{k}_{a} \\ C L \\ V\end{matrix} ] = [ \begin{matrix}{θ}_{1} {e}^{{η}_{i} , 3 {κ}_{i} , 1} \\ {{θ}_{2} ( \frac{{wt}_{i}}{70} )}^{0.75} {θ}_{2}^{s e {x}_{i}} {e}^{{η}_{i} , 1} \\ {θ}_{3} {e}^{{η}_{i} , 2}\end{matrix} ] \qquad ( 6 )$$

In this embodiment, the covariates are given by the vector Zᵢ in equation 7.

$${Z}_{i} = [ \begin{matrix}w {t}_{i} \\ s e {x}_{i}\end{matrix} ] \qquad ( 7 )$$

At each discrete time points tdₐ there is a dose event. Starting from =0, the model is an ODE on the time interval t∈[0, td₁]; then at that end time, another dose is administered that causes a discontinuous jump in uᵢ. The ODE is then integrated again over the time interval t∈[td₁, td₂], repeating until one hits the final time of a therapeutic time interval at t_{f}.

In various embodiments, a machine learning method is employed to estimate the θ parameters based on a dataset of measured concentrations in a population. Furthermore, in some embodiments, the model is updated as data from new patients becomes available, again using the same or different machine learning, and for an individual based on measured response to a dose administered to that individual.

Electronic health records (EHR) provide a rich dataset for employing machine learning methods. Thus, in various embodiments, numerical solutions for the differential equations of a NLME model are implemented as an activation layer of a neural net architecture. By directly encoding the ODE dynamics and events into the neural net layer via its activation function, one can retain the properties of the modeling-based approach so that the outputted values (the predicted concentrations) are biologically realistic and follow the proposed clinical model. The population parameters and random distribution function are learned during this training, while maintaining the ODE relationships. In some embodiments, this dynamic layer is then embedded in an end to end neural network that has as input values and any measurements of therapeutic effects on an individual; and; has as an output layer the expected dose response and confidence in (probability of) the same. In other embodiments, other methods of determining the model θ parameters are used.

The parameters of this system would then be the input nodes of the dynamical layer, which are chained from earlier layers of a neural network as predictions from arbitrary datasets. In this way, the relationship between electronic health records (EHR) records, imaging results, and molecular biological assays (protein assays, SNP arrays, RNA-seq) can be trained and deduced programmatically without a requiring a modeler assumption; directly generalizing previous PK/PD modeling approaches to contemporary big data.

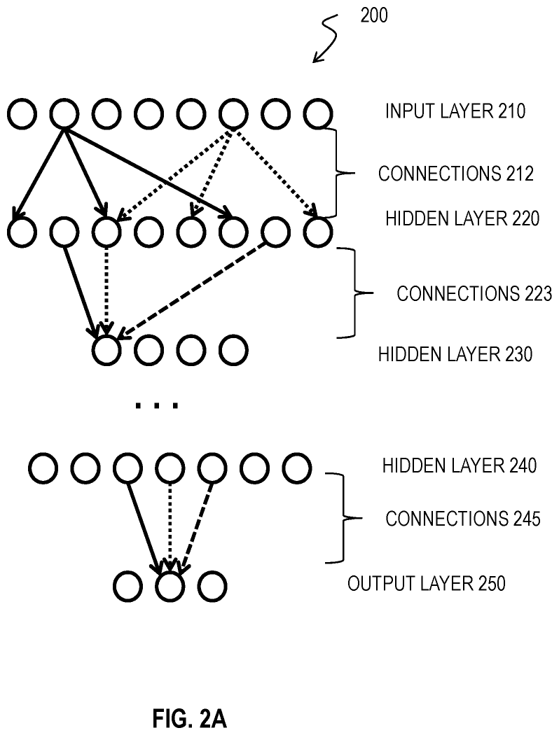

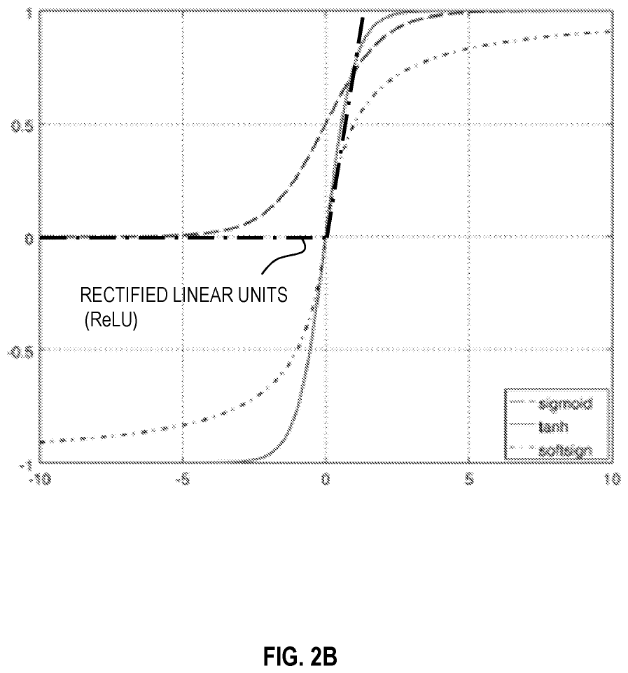

FIG. 2A is a block diagram that illustrates example flow structure of a generic neural network computation. A neural network is a computational system, implemented on a general-purpose computer or application specific integrated circuit (ASIC), which is made up of an input layer of nodes, at least one hidden layer of nodes, and an output layer of one or more nodes. Each node is an element, such as a register or memory location, that holds data that indicates a value. The value can be code, binary, integer, floating point or any other means of representing data. Values in nodes in each successive layer after the input layer in the direction toward the output layer is based on the values of one or more nodes in the layer before. The nodes in one layer that contribute to the next layer are said to be connected to the node in the later layer. The values of the connected nodes are combined at the node in the later layer using some activation function with scale and bias (also called weights) that can be different for each connection. Neural networks are so named because they are modeled after the way neuron cells are connected in biological systems. A fully connected neural network has every node at each layer connected to every node at any previous or later layer.

For example, the neural network 200 depicted in FIG. 2A has an input layer 210 of eight nodes, each node represented by an open circle. The network 200 includes hidden layers 220, 230 and 240, among zero or more others indicated by ellipsis, and an output layer 250 of three nodes. The output layer nodes hold values that represent the result of the neural network computation for three parameters. FIG. 2A also shows a few example connections as arrows connecting a node in one layer with one or more nodes in a later layer. All connections inherent in a fully connected version of the depicted network are not shown in order to avoid obfuscating the illustration. One node in input layer 210 is connected to three nodes in hidden layer 220 by three connections represented by solid arrows; and, another node in input layer 210 is connected to three nodes in hidden layer 220 by three connections represented by three dotted arrows. In other embodiments each node in one layer is connected to more or fewer nodes in the next layer. Each node in a later layer thus has a value based on all the nodes that are connected to it, as illustrated for one node in hidden layer 230, which has a value based on a combination of values in three nodes in the previous layer, represented by a solid arrow, dotted arrow and dashed arrow. In other embodiments, each node in one layer combines values from more or fewer nodes in the previous layer.

FIG. 2B is a plot that illustrates example activation functions used to combine inputs at any node of a neural network. These activation functions are normalized to have a magnitude of 1 and a bias of zero; but when associated with any connection can have a variable magnitude given by a weight and centered on a different value given by a bias. The values in the output layer 250 depend on the activation functions used at each node and the weights and biases associated with each connection that terminates on that node. The sigmoid activation function (dashed trace) has the property that values much less than the center value do not contribute to the combination (a so called switch off effect) and large values do not contribute more than the maximum value to the combination (a so-called saturation effect), both properties frequently observed in natural neurons. The tan h activation function has similar properties but allows both positive and negative contributions. The softsign activation function is similar to the tan h function but has much more gradual switch and saturation responses. The rectified linear units (ReLU) activation function simply ignores negative contributions from nodes on the previous layer; but, increases linearly with positive contributions from the nodes on the previous layer; thus, ReIU activation exhibits switching but does not exhibit saturation. In some embodiments, the activation function operates on individual connections before a subsequent operation, such as summation or multiplication; in other embodiments, the activation function operates on the sum or product of the values in the connected nodes. In other embodiments, other activation functions are used, such as kernel convolution and embodiments of certain equations of any models being implemented within the network, including differential or integral equations.

An advantage of neural networks is that they can be trained to produce a desired output from a given input without knowledge of how the desired output is computed. There are various algorithms known in the art to train the neural network on example inputs with known outputs. Typically, the activation function for each node or layer of nodes is predetermined, and the training determines the weights and biases for each connection. A trained network that provides useful results, e.g., with demonstrated good performance for known results, is then used in operation on new input data not used to train or validate the network.

In image processing, it was found that neural networks configured to perform convolution over relatively small two-dimensional portions of an image, called the receptive field, provided superior results with smaller networks. Here each input layer node corresponds to one or more pixels or voxels in an input image. Such neural networks are called convolution neural networks (CNN) and are modeled after the neurons in the visual cortex of animals. As in the biological models, receptive fields of different neurons partially overlap such that they cover the entire visual field. In such neural networks, not only the activation function, but also the weights and biases, are shared for an entire layer. This provides the networks with shift and rotation invariant responses. The hidden layers of a CNN typically consist of convolutional layers, pooling layers, fully connected layers and normalization layers. The convolutional layer has parameters made up of a set of learnable filters (or kernels), which have a small receptive field (e.g., 3×3). The filter is used for one or more of blurring, sharpening, embossing, edge detection, among others. This is accomplished by doing a convolution between the filter and the receptive field. In a convolution layer, each filter is convolved across the width and height of the previous layer, e.g., the input image, computing the dot product between the entries of the filter and the previous and producing a 2-dimensional activation map of that filter. The same kernel, weights and biases are used for every node in the layer. In a pooling layer, the activation functions perform a form of non-linear down-sampling, e.g., producing one node with a single value to represent four nodes in a previous layer. There are several non-linear functions to implement pooling among which max pooling is the most common. A normalization layer simply rescales the values in a layer to lie between a predetermined minimum value and maximum value, e.g., 0 and 1, respectively.

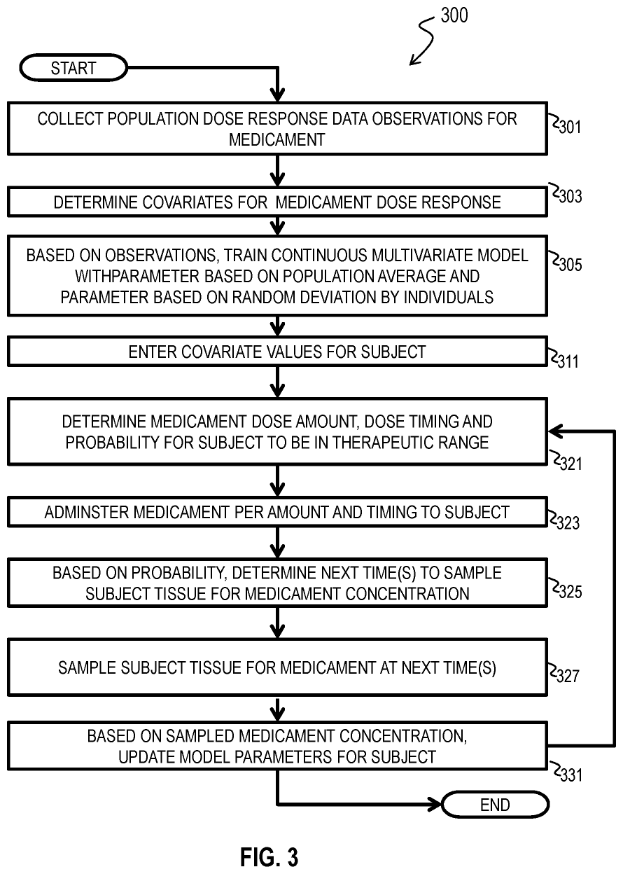

FIG. 3 is flow diagram that illustrates an example method for objectively and automatically determining model parameters and updating a model using medicament response observations for an individual during treatment, according to an embodiment. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, population dose response data for a particular medicament is collected, e.g., by sampling or sub-sampling EHR for patients receiving the medicament for test or treatment.

In step 303 various covariates are determined for the dose response based on analysis of the data collected in step 301 or prior knowledge. For example, for vancomycin, a rate of glomerular filtration (associated with a cluster of nerve endings, spores, or small blood vessels, especially around the end of a kidney tubule) was found to be a covariate with clearance, CL, over the population. More details on this vancomycin model are described in the Example Embodiments section.

In step 305, a NLME model is developed with appropriate random and nonrandom parameters; and, the model parameters are learned using methods of machine learning and a training set, such as training a neural network on historical data in the EHR. More details on the vancomycin model and its training are described in the Example Embodiments section. The training continues until a certain stop condition is achieved, such as a number of iterations, or a maximum or percentile difference between model output and measured values is less than some tolerance, or a difference in results between successive iterations is less than some target tolerance, or the results begin to diverge, or other known measure, or some combination. As a result of the training, the model is a continuous multivariate model that requires no classification or binning of the subject be performed, as is required in the application of the nomograms of the prior art. As a result of step 305, a continuous multivariate model is received on a processor as first data that indicates, for dose response to a medicament, with at least one population parameter based on a single value that characterizes a population of individuals and at least one random parameter based on a distribution of values for individuals in the population.

In step 311, an individual to be treated with the medicament is identified; and, the values of the covariates for that individual are determined. For example, a patient ID is provided to the system along with values of the covariates for the individual, such as the gender, age, weight, and glomerular filtration rate measured or inferred for the patient. Any method may be used to enter this information. For example, it, and other inputs described herein, may be input manually, may be retrieved automatically or in response to a command from storage or other computer-readable medium, in a distributed or integrated database or flat files, either locally or remotely, either in response to a request or unsolicited, or any other manner known in the art.

In step 321, the model is run to determine a dose amount and dose timing for each of one or more dose administrations and a probability for the individual to be in the target therapeutic range. This determination is based on the values of the covariates for the individual subject and the relations of proper dose for a subject with those covariate values in the population. In some embodiments the probability is based on a set of random samples selected from each of the random parameter distributions. Because there is no measurement of the individual's particular response to the medicament, the resulting dose recommendations are considered naïve, and the probability of attaining the target therapeutic dose can be relatively low. For example, using a naive vancomycin dosing protocol, even from the continuous multivariate model described above, and using current clinical practice of widely timed measurements of samples from the subject, less than 45% of subjects maintain a concentration in the therapeutic range for a therapeutic period.

In step 323 the medicament is administered to the subject per the dose and timing recommended by the model. Thus, a first dose of the medicament is administered to a subject based on the model and a naïve most probable value for the at least one random parameter.

In step 325, based on the probability of the subject having a concentration in the target therapeutic range, zero or more measurement times are determined to sample tissue of the subject for concentration of the medicament or other therapeutic effect, such as tumor size change. The lower the probability that the subject will have a therapeutic effect in the therapeutic range, the more frequent and the more closely spaced are the measurement times. This step is in contrast to previous methods, in which the next measurement times were based on population averages and not based on data from the individual being treated or during treatment. Thus step 325 includes determining, automatically on the processor based on the model, a probability that the first dose will produce a therapeutic result and a set of one or more times to sample the subject for corresponding measures of the therapeutic effect based on the probability. In some embodiments, the measurements times are determined to be at least two measurements between the first two doses in the naïve protocol. By definition, the naïve prediction is that the concentration is in the effective range during the time interval between the two doses. It is advantageous for estimating an actual subject-specific elimination rate, to have the two samples in the time interval not too close together. Thus, the sample measurement times are often expressed in terms of the end points of the interval. For example, in some embodiments, the sampling recommendation is at least one hour after the first dose is given and at least one hour before the end of the time interval, when the second dose is given. When the probabilities of remaining in the therapeutic range are not sufficiently high, say >90%, the same sampling recommendation is made, no matter which dosing interval is current. At later dosing intervals, the probability may improve sufficiently that no new sampling times are recommended.

In step 327, a sample is taken from the subject to determine therapeutic effect (e.g., concentration of the medicament in the tissue of the sample) at the zero or more measurement times determined during step 325. This step is performed in a timely way to correct any deficiencies in administering the medicament before harmful effects accrue to the subject, such as ineffective or toxic results. This step is in contrast to previous methods, in which the next dose of the medicament is administered regardless of whether the first dose was ineffective or toxic for the individual subject. Thus, step 327 includes sampling the subject at the set of one or more times to obtain corresponding values of the corresponding measures of the therapeutic effect.

In step 331 the model determined in step 305 is updated, if at all, based on the measurements taken in step 327. For example, if the medicament concentration value is less than or greater than the therapeutic range, it is determined that the individual has a response that deviates substantively from the population average values. Any method may be used to update the model parameters to fit the observed response of the individual, such as new training of the neural network. In some embodiments, the population model is updated based on the one observation of the individual being treated in proportion to the number of different subjects that previously contributed to the model. In some embodiments, in addition to or instead of updating the population model parameter values, special subject-specific model parameter values are generated.

Various method may be used to update the parameters of the model based on the measurements. To move the population model toward a subject-specific model that better suits the one subject, either a neural network or other machine learning approach can be re-trained with a training set that multiplies the weight of the current subject relative to all other data in the model, e.g., by a factor of 2, 5, 25, 100, 1,000, 10,000 or more. In some embodiments, the random distributions are modified, so that samples closer to the measured values are obtained during the forward operation of the model. The model, with its learned parameters, serves as the prior knowledge. The subject-specific concentrations serve as the likelihood used to generate the subject specific posterior distribution of possible parameters. As a result, a parameter, such as the elimination rate in the plasma, is updated an may differ from the population average value. This posterior distribution of parameter values then is used to project the new dose and dosing regimen, including dosing intervals for that subject. The posterior distribution is used to estimate the probability of remaining in the therapeutic range throughout the dosing interval. With each new set of measurements, the cycle is repeated until the confidence in the recommended dosing regimen is high. Thus, step 331 includes determining, automatically on the processor based on the model, a subject-specific most probable value for the at least one random parameter based on the corresponding values.

Control then passes to repeat steps 321 to 331 with the updated population model or the subject-specific model for the subject being treated. It is expected that at each repeat through the loop from steps 221 through 331, the probability of the subject having an effect within the target therapeutic range increases (i.e., probability of the subject-specific most probable value of the random parameters is greater than a probability of the naive most probable value of the random parameter); and, thus, that the number or frequency of measurement times, or both, is reduced. Thus, the second time through the loop, step 321 includes determining, automatically on the processor based on the model, a second dose and timing therefor based on the subject-specific most probable value.

The method 300 thus determines, during treatment of a subject, individualized protocols for administering medicaments, which have increased chance to be maintained in the subject within a therapeutic range.

### 2. EXAMPLE EMBODIMENTS

According to an example embodiment, the method 300 is implemented for administering vancomycin to deliver and maintain a blood concentration within a therapeutic range.

Inventors previously developed a hierarchical Bayesian model to describe the population pharmacokinetics of vancomycin in neonates (26). Briefly, in step 305, a one-compartment linear disposition model with zero order input (one hour intravenous infusion) and first-order elimination was used to describe the data collected in step 301. In step 303 Weight (Wt) and estimated glomerular filtration rate (EGFR) were included as covariates on clearance (CL), and weight was included as covariate on volume of distribution (V). Back in step 305, a lognormal distribution was used to describe the between-subject-variability (BSV) in model parameter estimates CL and V, as expressed in Equation 8 and Equation 9.

$$\begin{matrix}C {L}_{i} = C {{L}_{t y p i c a l} ( \frac{Wt}{2.9} )}^{0.75} {( \frac{E G F R}{30} )}^{C L E G F R} {e}^{{η}_{C L}} & ( 8 ) \\ {V}_{i} = {V}_{typical} ( \frac{Wt}{2.9} ) {e}^{{η}_{V}} & ( 9 )\end{matrix}$$

where CLᵢ and Vᵢ are the true parameters for the individual, CL_{typical} and V_{typical} are the population value of the parameters, and η_{CL} and ηᵥ denote the deviation between the typical value of the parameters and the true parameter value of an individual. The coefficient that describes the relationship between EGFR and CL_{typical} is denoted by CLEGFR. The parameter estimates for the final population PK model are shown in Table 1, using Bayesian estimation. In Table 1, PM indicates posterior median; LCI indicates lower credible interval; and UCI indicates upper credible interval. In other embodiments, based on different datasets or different training approaches, the parameter values are expected to differ to some degree. In this embodiment, the values 2.9, 30, CL_{typical}, V_{typical} and CLEGFR are θ parameters.

TABLE 1
Population PK parameters of vancomycin in neonates. Final
population PK model Parameter Posterior Median (PM)
and 90% Credible Intervals (LCI and RCI)

| Population PK Parameter (unit) | PM | LCI | RCI |
|---|---|---|---|
| CL_{typical}^{a} (L/hr) | 0.167 | 0.163 | 0.17 |
| Exponent for WT (FIX) | 0.75 | — | — |
| Exponent for EGFR | 2.06 | 2.02 | 2.1 |
| V_{typical}^{b} (L) | 1.77 | 1.73 | 1.81 |
| Exponent for WT (FIX) | 1.0 | — | — |

Interindividual Variability

| | | | |
|---|---|---|---|
| CL (% CV) | 22.9 | 21.4 | 24.5 |
| CL V | 0.00277 | −0.00149 | 0.0074 |
| V (% CV) | 15.4 | 14.2 | 16.8 |

Residual Variability

| | | | |
|---|---|---|---|
| Proportional (% CV) | 17.3 | 16.8 | 17.9 |
| prop add | 0.00407 | −0.00754 | 0.015 |
| Additive (SD) | 1.18 | 1.07 | 1.32 |

^{a}typical individual-2.9 kg, estimated glomerular filtration rate of 30 mL/min per 1.73 m²
^{b}typical individual-2.9 kg

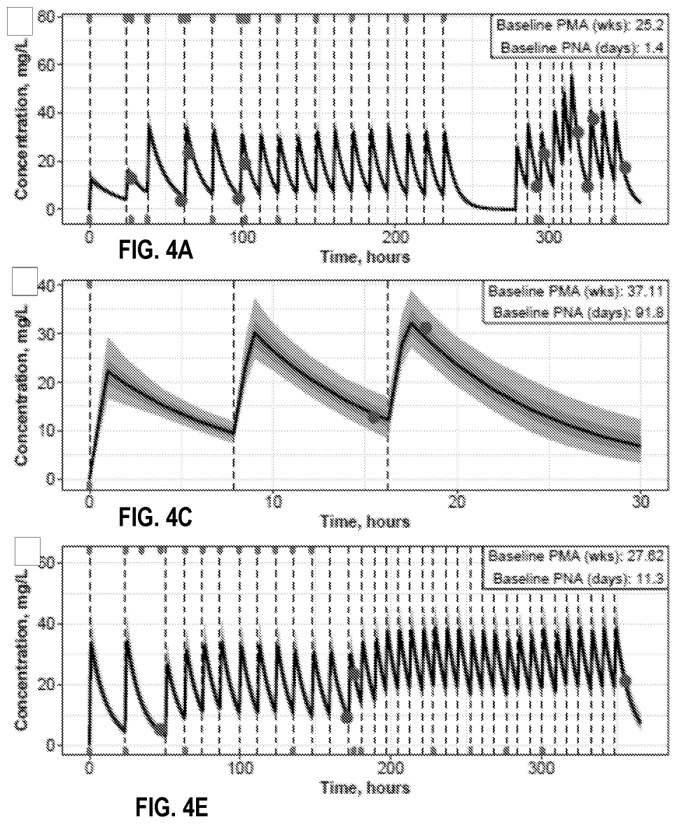

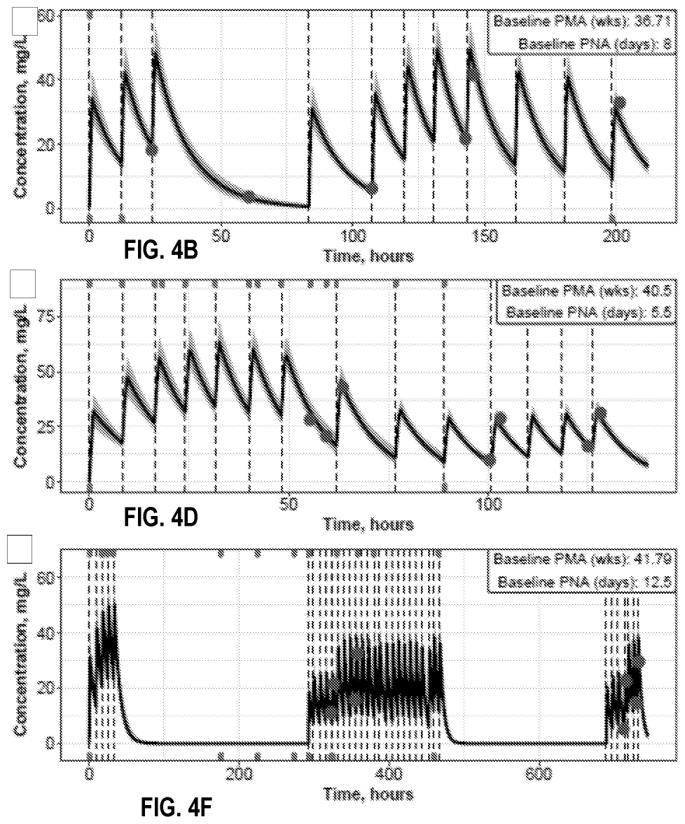

FIG. 4A through FIG. 4F are plots that illustrate example dose administration times, model results and measurements starting at time of first dose for six representative patients, according to an embodiment. FIG. 4A is for a patient who started treatment at 25.2 weeks PMA (post-menstrual age, indicating age after the mother's last menstrual event and thus the degree to which some of these patients are premature because a full term baby has a PMA of about 40 weeks) and 1.4 days PNA (post-natal age); FIG. 4C for at 37.11 weeks PMA and 91.8 days PNA; FIG. 4E for at 27.62 weeks PMA and 11.3 days PNA; FIG. 4B for at 36.71 weeks PMA and 8 days PNA; FIG. 4D for at 40.5 weeks PMA and 5.5 days PNA; and, FIG. 4F for at 41.79 weeks PMA and 12.5 days PNA. The horizontal axis indicates time in hours; and, the vertical axis indicates vancomycin concentration in milligram per liter (mg/L). Each plot provides the observations (dots) of vancomycin concentration, vertical dashed lines indicating a time of dose administration, and a trace of the model computed concentration, with bands indicating a range in which a measured concentration is 90% likely to fall (posterior predictive interval). Extra tick marks along the top of each plot indicates a serum creatinine measurement; and, extra tick marks along the bottom of each plot indicate a weight measurement.

Vancomycin dosing has standardized around a target antimicrobial index of 400 AUC0-24 h/MIC ratio, (27-29) which has been shown in adults to minimize risk of treatment failure. AUC refers to area under the concentration curve. So, technically AUC is the integral of concentrations over the time period. Thus, AUC is derived by integrating the concentration-time data depicted in FIG. 4A through FIG. 4F. However, given the practical difficulties of AUC derivation, and the fact that peak samples are infrequently taken in older populations, trough target levels have traditionally been used as a surrogate. An important assumption that is tied to the correlation between trough and exposure is that all patients receive dosing at the same interval. In neonates 6, 8, 12, 18, 24, and 48-hour intervals have all been reported. Generally, the longer intervals are given to younger patients, as they have less renal maturation to clear vancomycin. As peaks and troughs are still routinely taken in neonates, and many possible intervals are reasonable, it is vital to use the appropriate metric. Hence, an AUC/MIC ratio of 400 will be used for all dose recommendation calculations. However, the trough concentrations will also be tracked to compare against clinical practice.

The dose recommendation algorithm depicted in FIG. 3 can be delineated into two stages. The first time that step 321 is encountered, a naive recommendation is made in which only prognostic factors are available for an individual based on population statistics and covariate values. After measurements are made in step 327 and the model is updated in step 331, then when step 327 is next encountered, a tailored recommendation is made. The dose recommendation algorithm seeks to maximize the probability of target attainment (PTA), defined as the probability of a dosing regimen to achieve an AUC/MIC ratio greater than 400, weighted against the probability of concern (POC), defined as the probability of a dosing recommendation to achieve an AUC/MIC ratio of less than 400 (efficacy concern) or greater than 600 (safety concern, chosen hypothetically).

For a dose recommendation in a naive patient, only the patient's prognostic factors are available and potential clearance and volume values are generated from the full population PK model, the uncertainty of the potential range of exposures is initially dominated by interindividual variability, described by the η distributions. Hence, the range of exposures can be established from only the median marginal posterior estimates. Therefore, in step 321 in some embodiments, 250 individual clearance and volume parameterizations will be drawn from median posterior distribution estimates of the population model. To assess potential dosing regimens, in some embodiments, a grid of possible doses ranging from 5 mg/kg to 30 mg/kg, in two milligram increments, for each possible inter-dose interval (default of 8, 12, and 24 hours) is created. As the range of possible exposures in a naive patient is significantly broader than that of an individual patient, the naive algorithm balances probability of target attainment heuristics to account for this population-level variability, so to not inflate the dose recommendations too heavily, as larger doses will always increase PTA. Hence, for a naive patient, we propose a default weighting factor is 2:1 PTA:POC.

As data becomes available in an individual, the data is fit to the model in step 331, and a marginal posterior for each parameter obtained. A training validation data set can be used to simulate a scenario where data is available temporally and recommendations can be provided on the fly, similar to what is anticipated for real time EHR streaming.

In subsequent encounters with step 321, the updated model is used in the following ways. First, to thin unreasonable dosing regimens from the grid of possible doses and inter-dose intervals, all concentration-time profiles are simulated using the median marginal posterior values. Potential dosing regimens are retained if the daily exposure is between 400-600 mg/L*hr. For the retained dosing regimens, 250 parameter sets are drawn from the marginal posterior, concentration-time profiles generated, and steady-state daily exposures calculated. For each dosing regimen, the PTA is derived by calculating the number of times the exposure is greater than 400 divided by the total number of simulated profiles. Then, the best recommendation for each inter-dose interval is calculated by maximizing the probability of target attainment, while minimizing the probability of concern, where improvement in the probability of target attainment is weighted 4:1 over the probability of concern. That is to say, for a proposed regimen with a 1% improvement of probability of target attainment, a 4% increase in probability of concern would be needed to reject the proposal. For example, given two regimens, regimen A, with a probability of target attainment of 94% and a probability of concern of 15%, or regimen B, with a probability of target success of 96% and probability of concern of 28 percent, regimen A would be selected. After that selection, the best dosing regimens will be presented.

The dosing regimen proposed for the naive patients resulted in a 65% of dose recommendations within the target AU C0-24 hr/MIC range of 400-600. Addition of individual data to develop a dosing regimen recommendation from the generated marginal posteriors resulted in 94% target attainment with 2 observations, increasing to 98% as observations were added.

An AU C0-24 hr/MIC ratio of 600 was chosen as the cut-off as the recommended dose would be 50% higher than necessary, given the true parameter values were known. This was a conservative choice, given that AU C0-24 hr's exceeding 600 are currently seen in over 30% of patients, and is suitable for generalizing the algorithm. Vancomycin-induced nephrotoxicity is an acknowledged issue, however without more precise mechanisms to target dosing, safety considerations have been blunted in the quest for maximizing efficacy and reducing resistance development. This acceptability criteria was chosen as it is well within current exposures experienced, and would likely maximize efficacy while minimizing safety concerns. Furthermore, the target range is only one mechanism of tuning the algorithm. The weighting of safety vs efficacy concern can also be adjusted. Hence, 600 was chosen to represent a ‘failure.’

The dosing algorithm performed remarkably well. Overall, the ability to correctly propose future doses, and the distribution of true exposures given the proposed dosing regimen shows the prior specification is well tuned to identify appropriate marginal posteriors for individuals. Given two observations, the performance was entirely reliable within the test set.

### 3. COMPUTATIONAL HARDWARE OVERVIEW

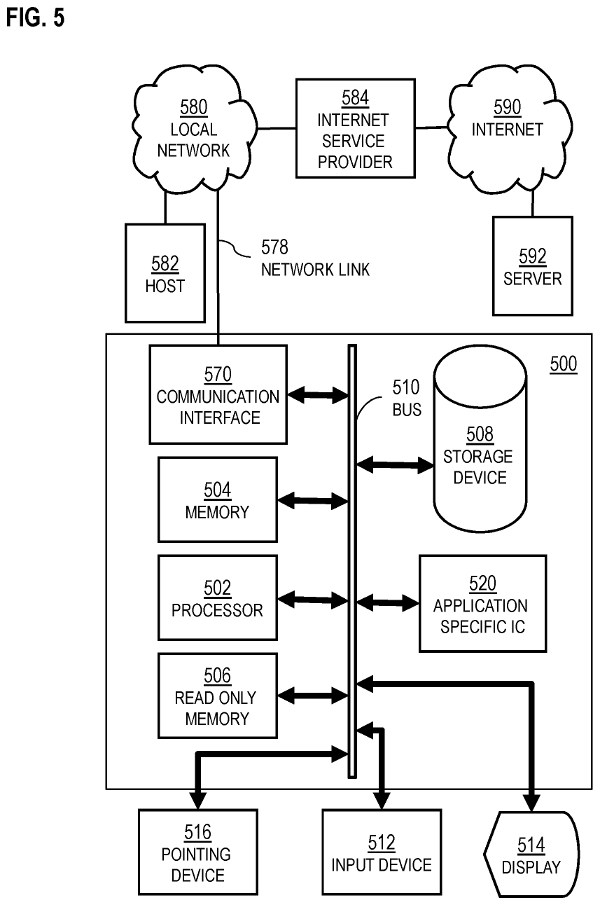

FIG. 5 is a block diagram that illustrates a computer system 500 upon which an embodiment of the invention may be implemented. Computer system 500 includes a communication mechanism such as a bus 510 for passing information between other internal and external components of the computer system 500. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 500, or a portion thereof, constitutes a means for performing one or more steps of one or more methods described herein.

A sequence of binary digits constitutes digital data that is used to represent a number or code for a character. A bus 510 includes many parallel conductors of information so that information is transferred quickly among devices coupled to the bus 510. One or more processors 502 for processing information are coupled with the bus 510. A processor 502 performs a set of operations on information. The set of operations include bringing information in from the bus 510 and placing information on the bus 510. The set of operations also typically include comparing two or more units of information, shifting positions of units of information, and combining two or more units of information, such as by addition or multiplication. A sequence of operations to be executed by the processor 502 constitutes computer instructions.

Computer system 500 also includes a memory 504 coupled to bus 510. The memory 504, such as a random access memory (RAM) or other dynamic storage device, stores information including computer instructions. Dynamic memory allows information stored therein to be changed by the computer system 500. RAM allows a unit of information stored at a location called a memory address to be stored and retrieved independently of information at neighboring addresses. The memory 504 is also used by the processor 502 to store temporary values during execution of computer instructions. The computer system 500 also includes a read only memory (ROM) 506 or other static storage device coupled to the bus 510 for storing static information, including instructions, that is not changed by the computer system 500. Also coupled to bus 510 is a non-volatile (persistent) storage device 508, such as a magnetic disk or optical disk, for storing information, including instructions, that persists even when the computer system 500 is turned off or otherwise loses power.

Information, including instructions, is provided to the bus 510 for use by the processor from an external input device 512, such as a keyboard containing alphanumeric keys operated by a human user, or a sensor. A sensor detects conditions in its vicinity and transforms those detections into signals compatible with the signals used to represent information in computer system 500. Other external devices coupled to bus 510, used primarily for interacting with humans, include a display device 514, such as a cathode ray tube (CRT) or a liquid crystal display (LCD), for presenting images, and a pointing device 516, such as a mouse or a trackball or cursor direction keys, for controlling a position of a small cursor image presented on the display 514 and issuing commands associated with graphical elements presented on the display 514.

In the illustrated embodiment, special purpose hardware, such as an application specific integrated circuit (IC) 520, is coupled to bus 510. The special purpose hardware is configured to perform operations not performed by processor 502 quickly enough for special purposes. Examples of application specific ICs include graphics accelerator cards for generating images for display 514, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 500 also includes one or more instances of a communications interface 570 coupled to bus 510. Communication interface 570 provides a two-way communication coupling to a variety of external devices that operate with their own processors, such as printers, scanners and external disks. In general the coupling is with a network link 578 that is connected to a local network 580 to which a variety of external devices with their own processors are connected. For example, communication interface 570 may be a parallel port or a serial port or a universal serial bus (USB) port on a personal computer. In some embodiments, communications interface 570 is an integrated services digital network (ISDN) card or a digital subscriber line (DSL) card or a telephone modem that provides an information communication connection to a corresponding type of telephone line. In some embodiments, a communication interface 570 is a cable modem that converts signals on bus 510 into signals for a communication connection over a coaxial cable or into optical signals for a communication connection over a fiber optic cable. As another example, communications interface 570 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 570 sends and receives electrical, acoustic or electromagnetic signals, including infrared and optical signals, that carry information streams, such as digital data.

The term computer-readable medium is used herein to refer to any medium that participates in providing information to processor 502, including instructions for execution. Such a medium may take many forms, including, but not limited to, non-volatile media, volatile media and transmission media. Non-volatile media include, for example, optical or magnetic disks, such as storage device 508. Volatile media include, for example, dynamic memory 504. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 502, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 502, except for carrier waves and other signals.

Logic encoded in one or more tangible media includes one or both of processor instructions on a computer-readable storage media and special purpose hardware, such as ASIC 520.

Network link 578 typically provides information communication through one or more networks to other devices that use or process the information. For example, network link 578 may provide a connection through local network 580 to a host computer 582 or to equipment 584 operated by an Internet Service Provider (ISP). ISP equipment 584 in turn provides data communication services through the public, world-wide packet-switching communication network of networks now commonly referred to as the Internet 590. A computer called a server 592 connected to the Internet provides a service in response to information received over the Internet. For example, server 592 provides information representing video data for presentation at display 514.

The invention is related to the use of computer system 500 for implementing the techniques described herein. According to one embodiment of the invention, those techniques are performed by computer system 500 in response to processor 502 executing one or more sequences of one or more instructions contained in memory 504. Such instructions, also called software and program code, may be read into memory 504 from another computer-readable medium such as storage device 508. Execution of the sequences of instructions contained in memory 504 causes processor 502 to perform the method steps described herein. In alternative embodiments, hardware, such as application specific integrated circuit 520, may be used in place of or in combination with software to implement the invention. Thus, embodiments of the invention are not limited to any specific combination of hardware and software.

The signals transmitted over network link 578 and other networks through communications interface 570, carry information to and from computer system 500. Computer system 500 can send and receive information, including program code, through the networks 580, 590 among others, through network link 578 and communications interface 570. In an example using the Internet 590, a server 592 transmits program code for a particular application, requested by a message sent from computer 500, through Internet 590, ISP equipment 584, local network 580 and communications interface 570. The received code may be executed by processor 502 as it is received, or may be stored in storage device 508 or other non-volatile storage for later execution, or both. In this manner, computer system 500 may obtain application program code in the form of a signal on a carrier wave.

Various forms of computer readable media may be involved in carrying one or more sequence of instructions or data or both to processor 502 for execution. For example, instructions and data may initially be carried on a magnetic disk of a remote computer such as host 582. The remote computer loads the instructions and data into its dynamic memory and sends the instructions and data over a telephone line using a modem. A modem local to the computer system 500 receives the instructions and data on a telephone line and uses an infra-red transmitter to convert the instructions and data to a signal on an infra-red a carrier wave serving as the network link 578. An infrared detector serving as communications interface 570 receives the instructions and data carried in the infrared signal and places information representing the instructions and data onto bus 510. Bus 510 carries the information to memory 504 from which processor 502 retrieves and executes the instructions using some of the data sent with the instructions. The instructions and data received in memory 504 may optionally be stored on storage device 508, either before or after execution by the processor 502.

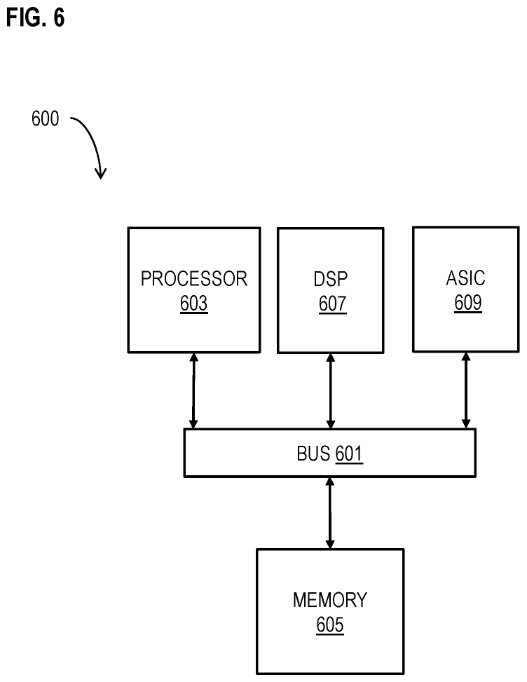

FIG. 6 illustrates a chip set 600 upon which an embodiment of the invention may be implemented. Chip set 600 is programmed to perform one or more steps of a method described herein and includes, for instance, the processor and memory components described with respect to FIG. 5 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 600, or a portion thereof, constitutes a means for performing one or more steps of a method described herein.

In one embodiment, the chip set 600 includes a communication mechanism such as a bus 601 for passing information among the components of the chip set 600. A processor 603 has connectivity to the bus 601 to execute instructions and process information stored in, for example, a memory 605. The processor 603 may include one or more processing cores with each core configured to perform independently. A multi-core processor enables multiprocessing within a single physical package. Examples of a multi-core processor include two, four, eight, or greater numbers of processing cores. Alternatively, or in addition, the processor 603 may include one or more microprocessors configured in tandem via the bus 601 to enable independent execution of instructions, pipelining, and multithreading. The processor 603 may also be accompanied with one or more specialized components to perform certain processing functions and tasks such as one or more digital signal processors (DSP) 607, or one or more application-specific integrated circuits (ASIC) 609. A DSP 607 typically is configured to process real-world signals (e.g., sound) in real time independently of the processor 603. Similarly, an ASIC 609 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 603 and accompanying components have connectivity to the memory 605 via the bus 601. The memory 605 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 605 also stores the data associated with or generated by the execution of one or more steps of the methods described herein.

### 4. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 5. REFERENCES

The following citations are referenced in the above.
1. Darwich A S, Ogungbenro K, Vinks A A, Powell J R, Reny J-L, Marsousi N, et al. Why has model-informed precision dosing not yet become common clinical reality? Lessons from the past and a roadmap for the future. Clin Pharmacol Ther [Internet]. 2017 February [cited 2017 Mar. 15]; Available from: http://doi.wiley.com/10.1002/cpt.659
2. Leroux S, Jacqz-Aigrain E, Biran V, Lopez E, Madeleneau D, Wallon C, et al. Clinical Utility and Safety of a Model-Based Patient-Tailored Dose of Vancomycin in Neonates. Antimicrob Agents Chemother. 2016 Apr. 1; 60(4):2039-42.
3. Bezanson J, Edelman A, Karpinski S, Shah V. Julia: A Fresh Approach to Numerical Computing. SIAM Rev. 2017 Jan. 1; 59(1):65-98.
4. Computing J. Big Time Series Analysis with Julia D B. Wilmott. 2018(95):10-3.
5. Rackauckas C, Nie Q. Differential Equations.jl—A Performant and Feature-Rich Ecosystem for Solving Differential Equations in Julia. J Open Res Softw [Internet]. 2017 May 25 [cited 2018 May 28]; 5(1). Available from: http://openresearchsoftware.metajnl.com/articles/10.5334/jors.151/
6. Wu P Y, Cheng C W, Kaddi C D, Venugopalan J, Hoffman R, Wang M D. #x2013; Omic and Electronic Health Record Big Data Analytics for Precision Medicine. IEEE Trans Biomed Eng. 2017 February; 64(2):263-73.
7. McQueen K, Murphy-Oikonen J. Neonatal Abstinence Syndrome. N Engl J Med. 2016 Dec. 22; 375(25):2468-79.
8. Pryor J R, Maalouf F I, Krans E E, Schumacher R E, Cooper W O, Patrick S W. The opioid epidemic and neonatal abstinence syndrome in the USA: a review of the continuum of care. Arch Dis Child—Fetal Neonatal Ed. 2017 Mar. 1; 102(2):F183-7.
9. Reddy U M, Davis J M, Ren Z, Greene M F, Opioid Use in Pregnancy, Neonatal Abstinence Syndrome, and Childhood Outcomes Workshop Invited Speakers. Opioid Use in Pregnancy, Neonatal Abstinence Syndrome, and Childhood Outcomes: Executive Summary of a Joint Workshop by the Eunice Kennedy Shriver National Institute of Child Health and Human Development, American College of Obstetricians and Gynecologists, American Academy of Pediatrics, Society for Maternal-Fetal Medicine, Centers for Disease Control and Prevention, and the March of Dimes Foundation. Obstet Gynecol. 2017 July; 130(1):10-28.
10. Ko J Y. Incidence of Neonatal Abstinence Syndrome—28 States, 1999-2013. MMWR Morb Mortal Wkly Rep [Internet]. 2016 [cited 2018 May 28]; 65. Available from: https://www.cdc.gov/mmwr/volumes/65/wr/mm6531a2.htm
11. Patrick S W, Schumacher R E, Horbar J D, Buus-Frank M E, Edwards E M, Morrow K A, et al. Improving Care for Neonatal Abstinence Syndrome. Pediatrics. 2016 May; 137(5).
12. Kraft W K, Adeniyi-Jones S C, Chervoneva I, Greenspan J S, Abatemarco D, Kaltenbach K, et al. Buprenorphine for the Treatment of the Neonatal Abstinence Syndrome. N Engl J Med. 2017 Jun. 15; 376(24):2341-8.
13. Patrick S W, Schumacher R E, Benneyworth B D, Krans E E, McAllister J M, Davis M M. Neonatal abstinence syndrome and associated health care expenditures: United States, 2000-2009. JAMA. 2012 May 9; 307(18):1934-40.
14. Sanlorenzo L A, Stark A R, Patrick S W. Neonatal abstinence syndrome: an update. Curr Opin Pediatr. 2018 Jan. 17;
15. Agthe A G, Kim G R, Mathias K B, Hendrix C W, Chavez-Valdez R, Jansson L, et al. Clonidine as an adjunct therapy to opioids for neonatal abstinence syndrome: a randomized, controlled trial. Pediatrics. 2009 May; 123(5):e849-856.
16. Mould D R, Upton R N. Basic concepts in population modeling, simulation, and model-based drug development. CPT Pharmacomet Syst Pharmacol. 2012; 1:e6.
17. Mould D R, Upton R N. Basic Concepts in Population Modeling, Simulation, and Model-Based Drug Development—Part 2: Introduction to Pharmacokinetic Modeling Methods. CPT Pharmacomet Syst Pharmacol. 2013 April; 2(4):e38.
18. Jansson L M, Velez M, Harrow C. The opioid-exposed newborn: assessment and pharmacologic management. J Opioid Manag. 2009 February; 5(1):47-55.
19. Liu T, Lewis T, Gauda E, Gobburu J, Ivaturi V. Mechanistic Population Pharmacokinetics of Morphine in Neonates With Abstinence Syndrome After Oral Administration of Diluted Tincture of Opium. J Clin Pharmacol. 2016; 56(8):1009-18.
20. Liu T. Learn and Apply Paradigm to Optimize Pharmacotherapy in Neonatal Abstinence Syndrome Using Pharmacometrics [Internet]. 2017 [cited 2018 Feb. 14]. Available from: https://archive.hshsl.umaryland.edu/handle/10713/7395
21. Lacroix B D, Karlsson M O, Friberg L E. Simultaneous Exposure—Response Modeling of ACR20, ACR50, and ACR70 Improvement Scores in Rheumatoid Arthritis Patients Treated With Certolizumab Pegol. CPT Pharmacomet Syst Pharmacol. 2014 October; 3(10):e143.
22. Desrochers J, Wojciechowski J, Klein-Schwartz W, Gobburu J V S, Gopalakrishnan M. Bayesian Forecasting Tool to Predict the Need for Antidote in Acute Acetaminophen Overdose. Pharmacotherapy. 2017 August; 37(8):916-26.
23. Wright D F B, Duffull S B. Development of a Bayesian Forecasting Method for Warfarin Dose Individualisation. Pharm Res. 2011 May 1; 28(5):1100-11.
24. Jacqz-Aigrain E, Zhao W, Sharland M, van den Anker J N. Use of antibacterial agents in the neonate: 50 years of experience with vancomycin administration. Semin Fetal Neonatal Med. 2013 Feb. 1; 18(1):28-34.
25. Roberts J A, Aziz M H A, Lipman J, Mouton J W, Vinks A A, Felton T W, et al. Challenges and Potential Solutions—Individualised Antibiotic Dosing at the Bedside for Critically Ill Patients: a structured review. Lancet Infect Dis. 2014 June; 14(6):498-509.
26. Pastoor D. Innovation of vancomycin Treatment in neonates via A Bayesian dose Optimization toolkit for Adaptive individualized Therapeutic management [Internet]. 2017 [cited 2018 May 28]. Available from: https://bitly/2IW4BP5 27.
27. Rybak M J. Pharmacodynamics: Relation to Antimicrobial Resistance. Am J Med. 2006 Jun. 1; 119(6, Supplement 1):S37-44.
28. Frymoyer A, Hersh A L, Benet L Z, Guglielmo B J. Current Recommended Dosing of Vancomycin for Children with Invasive Methicillin-Resistant Staphylococcus aureus Infections is Inadequate. Pediatr Infect Dis J. 2009 May; 28(5):398-402.
29. Frymoyer A, Hersh A L, El-Komy M H, Gaskari S, Su F, Drover D R, et al. Association between Vancomycin Trough Concentration and Area under the Concentration-Time Curve in Neonates. Antimicrob Agents Chemother. 2014 Nov. 1; 58(11):6454-61.
30. Ivaturi V, Dvorak C C, Chan D, Liu T, Cowan M J, Wahlstrom J, et al. Pharmacokinetics and Model-Based Dosing to Optimize Fludarabine Therapy in Pediatric Hematopoietic Cell Transplant Recipients. Biol Blood Marrow Transplant J Am Soc Blood Marrow Transplant. 2017 October; 23(10):1701-13.

What is claimed is:

1. A method for generating a dosing protocol for an individual, the method comprising:
receiving on a processor first data that indicates, for dose response to a medicament, a continuous multivariate model with at least one population parameter based on a single value that characterizes a population of individuals and at least one random parameter based on a distribution of values for individuals in the population;
administering to a subject a first dose of the medicament based on the model and a naïve most probable value for the at least one random parameter;
determining, automatically on the processor based on the model, a probability that the first dose will produce a therapeutic result and a set of one or more times to sample the subject for corresponding measures of the therapeutic effect based on the probability;
sampling the subject at the set of one or more times to obtain corresponding values of the corresponding measures of the therapeutic effect;
determining, automatically on the processor based on the model, a subject-specific most probable value for the at least one random parameter based on the corresponding values; and
determining, automatically on the processor based on the model, a second dose and timing therefor based on the subject-specific most probable value.

2. The method of claim 1, further comprising administering to the subject the second dose of the medicament.

3. The method of claim 1, wherein the at least one random parameter is based on a distribution of values in electronic health records (EHR).

4. The method of claim 1, wherein the at least one random parameter is at least one of clearance or volume.

5. The method of claim 1, wherein the at least one population parameter is learned based on machine learning and a training set based on electronic health records (EHR).

6. The method of claim 5, wherein the machine learning is based on a neural network.

7. The method of claim 1, wherein the continuous multivariate model includes at least one covariate variable and the method further comprises receiving on a processor second data that indicates a corresponding value for the at least one covariate variable.

8. The method of claim 7, wherein the at least one covariate variable is at least one of weight or age or gender.

9. The method of claim 1, wherein the medicament is vancomycin.

10. The method of claim 7, wherein the medicament is vancomycin and the at least one covariate variable includes estimated glomerular filtration rate.

11. The method of claim 1, where a probability of the subject-specific most probable value is greater than a probability of the naive most probable value.

12. A non-transitory computer-readable medium carrying one or more sequences of instructions for generating a dosing protocol for an individual, wherein execution of the one or more sequences of instructions by one or more processors causes the one or more processors to perform the steps of:
receiving first data that indicates, for dose response to a medicament, a continuous multivariate model with at least one population parameter based on a single value that characterizes a population of individuals and at least one random parameter based on a distribution of values for individuals in the population;
determining, based on the model, a probability that a first dose based on the model and a naive most probable value for the at least one random parameter will produce a therapeutic result and a set of one or more times to sample the subject for corresponding measures of the therapeutic effect based on the probability;
receiving second data that indicates at the set of one or more times corresponding values of the corresponding measures of the therapeutic effect;
determining, based on the model, a subject-specific most probable value for the at least one random parameter based on the corresponding values; and
determining, based on the model, a second dose and timing therefor based on the subject-specific most probable value.

13. The computer-readable medium of claim 12, wherein the at least one random parameter is based on a distribution of values in electronic health records (EHR).

14. The computer-readable medium of claim 12, wherein the at least one random parameter is at least one of clearance or volume.

15. The computer-readable medium of claim 12, wherein the at least one population parameter is learned based on machine learning and a training set based on electronic health records (EHR).

16. The computer-readable medium of claim 15, wherein the machine learning is based on a neural network.

17. The computer-readable medium of claim 12, wherein: the continuous multivariate model includes at least one covariate variable; and, execution of the one or more sequences of instructions by the one or more processors further causes the one or more processors to perform the step of receiving on a processor second data that indicates a corresponding value for the at least one covariate variable.

18. The computer-readable medium of claim 17, wherein the at least one covariate variable is at least one of weight or age or gender.

19. The computer-readable medium of claim 12, wherein the medicament is vancomycin.

20. The computer-readable medium of claim 17, wherein the medicament is vancomycin and the at least one covariate variable includes estimated glomerular filtration rate.

21. An apparatus for generating a dosing protocol for an individual, the apparatus comprising:
at least one processor; and
at least one memory including one or more sequences of instructions,
the at least one memory and the one or more sequences of instructions configured to, with the at least one processor, cause the apparatus to perform at least the following:
receive first data that indicates, for dose response to a medicament, a continuous multivariate model with at least one population parameter based on a single value that characterizes a population of individuals and at least one random parameter based on a distribution of values for individuals in the population;
determine, based on the model, a probability that a first dose based on the model and a naive most probable value for the at least one random parameter will produce a therapeutic result and a set of one or more times to sample the subject for corresponding measures of the therapeutic effect based on the probability;
receive second data that indicates at the set of one or more times corresponding values of the corresponding measures of the therapeutic effect;
determine, based on the model, a subject-specific most probable value for the at least one random parameter based on the corresponding values; and
determine, based on the model, a second dose and timing therefor based on the subject-specific most probable value.
